(19) United States
(12) Patent Application Publication
(10) Pub. No.: US 2012/0226182 A1
(43) Pub. Date: Sep. 6, 2012

(54) DISPOSABLE THERMAL SENSOR FOR USE WITH A CANNULA

(75) Inventors: Richard Anthony BONATO, Carp (CA); David LOFSTROM, Ottawa (CA); Neil CAMERON, Ottawa (CA); Donald Carmon BRADLEY, Ottawa (CA); James Brent COWEN, Carleton Place (CA)

(73) Assignee: BRAEBON MEDICAL CORPORATION, Ottawa (CA)

(21) Appl. No.: 13/411,993

(22) Filed: Mar. 5, 2012

Related U.S. Application Data
(60) Provisional application No. 61/449,358, filed on Mar. 4, 2011.

(51) Int. Cl. A61B 5/01; A61B 5/08

(52) U.S. Cl. 600/537

(57) ABSTRACT

An airflow sensor (thermal sensor assembly) that is designed to adhesively attach to different styles of a cannula and that can detect the movement of respiratory air through the nasal and/or oral cavities. When secured to the cannula, the airflow sensor has its nasal and oral sensing elements in positions that will maximize signal accuracy, minimize airflow signal artifacts, and minimize occurrences of signal loss due to direct patient skin contact. The airflow sensor does not disturb the flow of air from the patient or add any discomfort to the patient. The airflow sensor can be attached to most nasal or nasal/oral cannulae used in sleep disorder diagnostics.

### CROSS REFERENCE TO RELATED APPLICATIONS

This application claims the benefit of priority of U.S. Provisional Patent Application No. 61/449,358 filed Mar. 4, 2011, which is incorporated herein by reference in its entirety.

### FIELD

The present disclosure relates generally to sleep disorder diagnostics. More particularly, the present disclosure relates to cannulae and airflow sensors used in sleep disorder diagnostics.

### BACKGROUND

Sleep apnea is characterized by a cessation or reduction of breathing that lasts at least 10 seconds and that is repeated at least 5 times an hour while the patient is sleeping. Obstructive sleep apnea (OSA) refers to apnea syndromes due primarily to collapse of the upper airway during sleep. It is estimated that 2 to 4% of middle aged people have OSA. OSA has two specific classifications of events: apnea and hypopnea. An apnea event is defined as an absence of airflow and a hypopnea event as a reduction in airflow associated with a blood oxygen reduction (desaturation) of 3 to 4%.

The American Academy of Sleep Medicine's Manual for the Scoring of Sleep and Associated Events©2007 (AASM) requires the use of an oral/nasal thermal sensor for the detection of apnea and a nasal air pressure transducer hypopnea. Both of these devices require the use of different technologies to measure the same physical phenomena, which is the movement of air in and out of the patient.

In the case of apnea, to measure nasal air pressure, it is standard to use a nasal cannula coupled to a pressure transducer. In the case of hypopnea, to measure air temperature, it is standard to mechanically attach the thermal sensor to the cannula. The coupling of the pressure transducer and the thermal sensor to the cannula can interfere with the patient's flow of air (i.e., can interfere with the patient's breathing), cause the thermal sensor to be deflected away from the flow of air (i.e., cause the thermal sensor to be misaligned with the flow of air), and have the thermal sensors actually come in contact with the patient's skin. All of these effects will cause errors in the thermal sensor signal, which can lead to incorrect diagnostics.

Example of known air flow sensors can be found in U.S. Pat. Nos. 5,558,099; 5,832,592; and 5,161,541 to Bowman et al. However, the air flow sensor assemblies in these references are adhered directly adhered to the patient's upper lip and do not allow the use of a nasal cannula, as required by the AASM for scoring hypopneas, without mutual interference between the cannula and the air flow sensor assemblies. The same issue exists in the disclosures of U.S. Pat. Nos. 5,311,875, 6,491,642 and 7,608,047 to Stasz and in the disclosure of U.S. Pat. Nos. 6,254,545 and 6,485,432 to Stasz et al. None of these prior references allow the use of the required cannula without either affecting the flow of air in or the patient's comfort.

With respect to diagnosing hypopnea, state of the art measurement requires the separate attachment of the thermal sensor to the cannula and of the cannula to the patient. This is a tedious and laborious task as the individual patient setup must secure the thermal sensor to the cannula, place both the cannula and the thermal sensor on the patient and then, secure the cannula and the thermal sensor on the patient (for example, by using adhesive tape). Securing the thermal sensor to the cannula must be made precisely and in relation to the patient: that is, the thermal sensor should be in the path of the airflow, the thermal sensor should not be touching any objects that can influence the sensors ability to sense the temperature of the airflow, the cannula should be centered on the nares of the patient, and the cannula should not be occluded by the thermal sensor. All this must be done just before the cannula and the thermal sensors are secured (taped down) to the patient.

Further, the sleep industry also uses combination nasal/oral cannulae as described in U.S. Pat. No. 7,337,780 to Curti et al, an in U.S. Design Pat. No. D559,383 to Nalagatla et al. These combination nasal/oral cannulae allow the measuring of both nasal and oral airflows. Such nasal/oral cannulae have nasal prongs to measure the nasal air pressure as well as some form of ducting that protrudes into or over the oral cavity. The combined use of oral thermal sensor and these nasal/oral cannulae would cause the oral thermal sensors to be ineffective in that they would occlude the ducting opening or would require the oral ducting on the cannula to be shifted in order for the oral thermal sensor to be properly positioned, which would cause the oral ducting to properly capture the oral airflow component.

Furthermore, sleep laboratories are looking towards medical devices that are single patient use for the diagnosis of OSA on patient's with highly infectious conditions. However, there are presently no acceptable single use thermal sensors for measuring apnea that can function properly in combination with a nasal or nasal/oral cannula. U.S. design Pat. Nos. D590,058 and D607,993 to Cowen show airflow sensors shaped to work with cannulae using existing concepts for reusable sensors. These designs will not allow the manufacturing of a cost effective device for single patient use.

Bowman, referenced above, and others disclose using an adhesive to hold the thermal sensor in place while on the patient. However, this requires the use of non aggressive medical adhesive. These thermal sensors cannot be placed on the cannula as this type of adhesive will not last the duration of a sleep study.

Several of the prior art references disclose the addition of an adhesive being applied that will attach the thermal sensor directly to the patient. However, the shape and the properties of the flexible substrate that is usually comprised in the thermal sensor do not allow for the thermal sensor to be easily attached to the patient.

Some prior art approaches allow for the placement of the thermal sensing element on top of a substrate. Such approaches require the thermal wave to pass through the substrate before reaching the sensor. This can lead to incorrect reading of the air temperature.

Therefore, improvements in thermal sensors for cannulae are desirable.

### SUMMARY

In a first aspect, the present disclosure provides a thermal sensor assembly to measure a temperature of air expelled by an individual, the thermal sensor assembly to be secured to a cannula having nasal prongs, the thermal sensor assembly secured to the cannula and the cannula secured to the individual defining an installed position. The thermal sensor assembly comprises: a substrate having a sensor portion, the substrate further having a sensor side and a backside, the backside being opposite the sensor side, the sensor portion defining an alignment aperture, the alignment aperture to receive at least one of the nasal prongs to align the thermal sensor assembly to the cannula; at least one nasal thermal sensor formed on the sensor side of the substrate and at the sensor portion of the substrate, the at least one nasal thermal sensor being adjacent to the alignment aperture, the at least one thermal sensor to sense, in the installed position, a temperature of air expelled through a nasal opening of the individual; and an adhesive layer formed on the backside of the substrate and at the sensor portion of the substrate, the adhesive layer to adhere the sensor portion of the substrate to the cannula.

In another aspect, the present disclosure provides a thermal sensor assembly to measure a temperature of air expelled by an individual, the thermal sensor assembly to be secured to a cannula having nasal prongs, the thermal sensor assembly secured to the cannula and the cannula secured to the individual defining an installed position. The thermal sensor assembly comprises: a substrate having a sensor portion, the substrate further having a sensor side and a backside, the backside being opposite the sensor side, the sensor portion defining an alignment feature, the alignment feature to receive at least one of the nasal prongs to align the thermal sensor assembly to the cannula; at least one nasal thermal sensor formed on the sensor side of the substrate and at the sensor portion of the substrate, the at least one nasal thermal sensor being adjacent to the alignment feature, the at least one thermal sensor to sense, in the installed position, a temperature of air expelled through a nasal opening of the individual; and an adhesive layer formed on the backside of the substrate and at the sensor portion of the substrate, the adhesive layer to adhere the sensor portion of the substrate to the cannula.

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached Figures.

FIG. 1 shows an embodiment of a thermal sensor assembly of the present disclosure secured to a cannula.

FIG. 2 shows a top view of the thermal sensor assembly of FIG. 1.

FIG. 3 shows a bottom view of the thermal sensor assembly of FIG. 1.

FIG. 4 shows a side view of the thermal sensor assembly of FIG. 1.

FIG. 5 shows a front view of the thermal sensor assembly of FIG. 1 being secured to a nasal portion of a cannula.

FIG. 6 shows a rear view of the thermal sensor assembly of FIG. 1 being secured to the nasal portion of a cannula.

FIG. 7 shows a front view of the thermal sensor assembly of FIG. 1 secured by a tab to a cannula.

FIG. 8 shows a rear view of the thermal sensor assembly of FIG. 1 secured to by a tab to a cannula.

FIG. 9 shows another front view of the thermal sensor assembly of FIG. 1 secured by a tab to a cannula.

FIG. 10 shows another rear view of the thermal sensor assembly of FIG. 1 secured to by a tab to a cannula.

FIG. 11 shows a front view of the thermal sensor assembly of FIG. 1 being secured to a nasal portion of an oronasal cannula.

FIG. 12 shows a rear view of the thermal sensor assembly of FIG. 1 being secured to a nasal portion of an oronasal cannula.

FIG. 13 shows a front view of the thermal sensor assembly of FIG. 1 secured by a tab to an oronasal cannula.

FIG. 14 shows a rear view of the thermal sensor assembly of FIG. 1 secured by a tab to an oronasal cannula.

FIG. 15 shows a front view of the thermal sensor assembly of FIG. 1 being secured by tabs to an oral section of an oronasal cannula.

FIG. 16 shows a rear view of the thermal sensor assembly of FIG. 1 being secured by tabs to an oral section of an oronasal cannula.

FIG. 17 shows a bottom view of another embodiment of a thermal sensor assembly in accordance with the present disclosure.

FIG. 18 shows a bottom view of another embodiment of a thermal sensor assembly in accordance with the present disclosure.

FIG. 19 shows a bottom view of another embodiment of a thermal sensor assembly in accordance with the present disclosure.

### DETAILED DESCRIPTION

Generally, the present disclosure provides a thermal sensor assembly that can be securely fixed to a cannula, in the correct position on the cannula, before the cannula is secured to the patient. The technician handling the cannula and the thermal sensor assembly only needs to be concerned about placing the cannula properly on the patient. The present disclosure allows for an easier and more accurate placement of the thermal sensor assembly and the cannula with respect to each other and with respect to the patient. Once the thermal sensor assembly is secured to the cannula, the technician simply has to tape the cannula in place, on the patient, and does need to be concerned about separately placing thermal sensors on the patient. The present disclosure allows for the placement of the thermal sensor directly in the path of the airflow. That is, there are no obstacles or materials between the thermal sensor and the flow of air. The present disclosure further allows the accurate placement of thermal sensors (thermal sensor assembly) on most nasal and oral/nasal cannulae presently on the market.

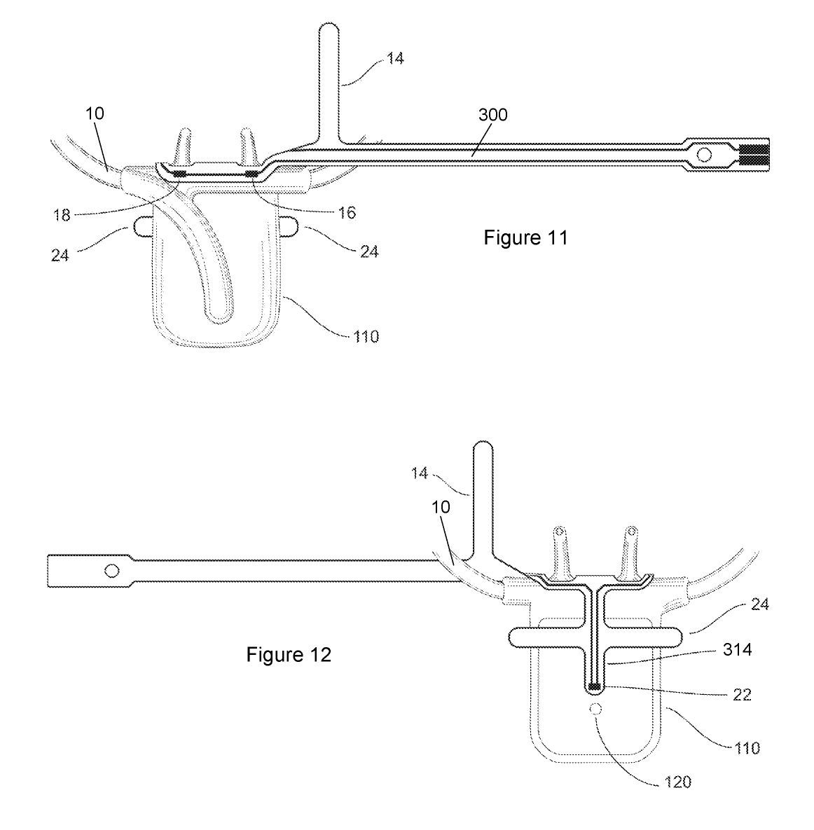

FIG. 1 shows an embodiment of a thermal sensor assembly 300 of the present disclosure. The thermal sensor assembly 300 is secured to a nasal cannula 10, which is secured to a patient 302. The thermal sensor assembly 300 has a sensor portion 308 that can be adhesively secured to the cannula 10 at the nasal portion 304. Similarly an oronasal cannula with an oral section 110 as shown at FIGS. 11 and 12 could have the oral section 20 of the sensor attached. The sensor portion 308 has nasal thermal sensors 16 and 18 positioned to receive air flowing out the nasal openings 306 of the patient 302. The cannula 10 has nasal prongs 307 inserted into the nasal openings 306 (nares). The nasal prongs 307 propagate air flowing out of the nasal openings 306 towards, for example, an air pressure monitor. The shape and size of nasal prongs 307 are such that only a portion of the air flowing out of the nasal openings 306 enters the nasal prongs 307. Another portion of the air flowing out of the nasal openings 306 impinges on the nasal thermal sensors 16 and 18. Additionally, the thermal sensor assembly 300 has a tail portion 8 and an intermediate portion 6 that physically connects the sensor portion 308 to the tail portion 8. As will be described in greater detailed below, the thermal sensor assembly 300 of FIG. 1 also has a tab 14 that can be used to further secure the thermal sensor assembly to the cannula 10.

The thermal sensor assembly 300 can comprise a thin, flexible non-electrically-conductive substrate (an electrically insulating substrate) such as, for example, mylar, polyester, and any other suitable type material that can be made thin and flexible.

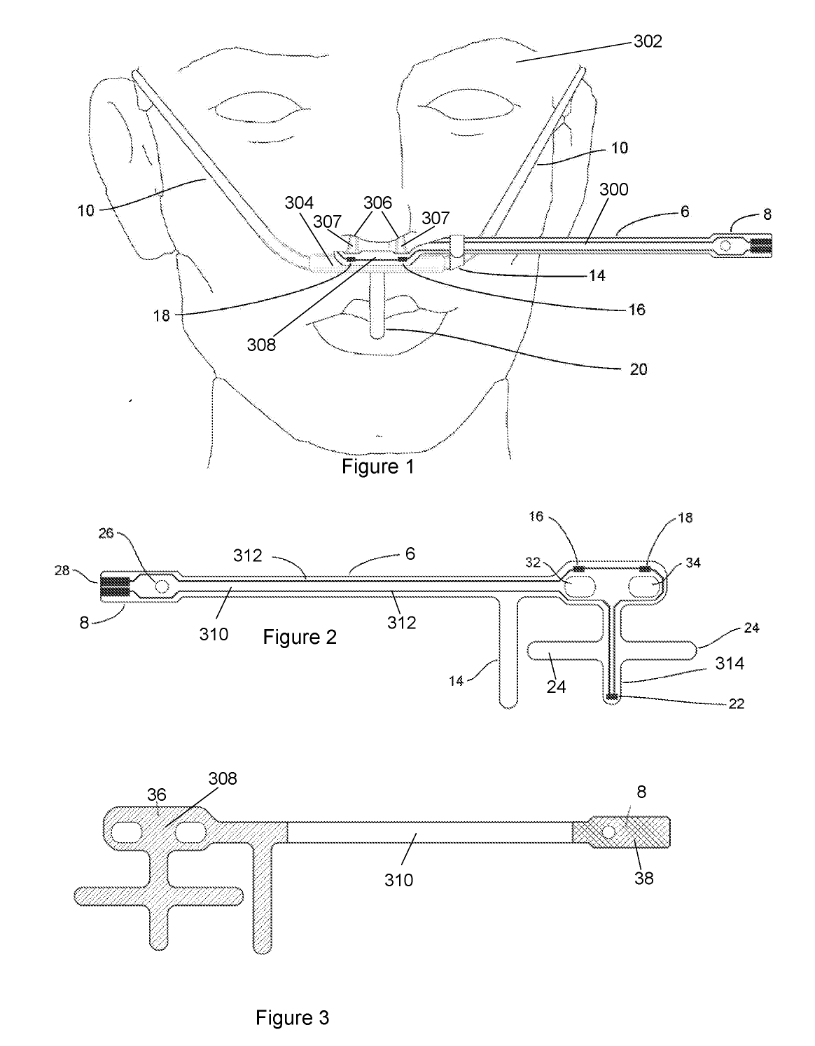

FIG. 2 shows a top view of the thermal sensor assembly 300 with such a substrate 310. The substrate 310 has electrically conductive traces 312 defined thereon. The electrically conductive traces 312 terminate at electrodes 28, which are defined at the tail portion 8 and which can be connected to any suitable measurement apparatus through any suitable connector arrangement. The electrical conductive traces 312 also electrically interconnect the nasal thermal sensors 16 and 18, as well as an oral thermal sensor 22, which can be secured to the oral section 120 of the cannula 10. The nasal and oral thermal sensors of the embodiment of FIG. 12 are electrically connected in series; however, any other type of electrical connection between the nasal and oral thermal sensors is also within the scope of the present disclosure. The electrically conductive traces 312 can include, for example, a conductive ink or any other suitable type of electrical conductor.

In another embodiment, instead of having two nasal thermal sensors 16 and 18, there can be only one nasal thermal sensor 17 that extends such as to receive air flowing out of either of the nasal openings 306.

The tail portion 8 can have defined therein a hole 26 that can be used to receive a cooperating part of a connector adapted to connect the electrodes 28 to the aforementioned measurement apparatus. The hole 26 receiving the cooperating part of the connector can help secure the electrodes 28, and the tail portion 8 to the connector.

The substrate 310 also defines the tab 14, which, as shown at FIG. 1, can be used to secure the thermal sensor assembly 300 to the cannula 10. The tab 14 is shown as extending perpendicularly from the intermediate portion 6; however, this need not be the case. For example, in another embodiment, the tab 14 can extend obliquely from the intermediate portion 6 and away from the sensor portion 308. Such an embodiment would also allow the oblique tab to secure the thermal sensor assembly as in the previous embodiment; however, in applications where it may be desired to remove the thermal assembly sensor 300 from the cannula 10, the oblique tab can facilitate the removal of the thermal sensor assembly 300 from the cannula 10 in that it can be easier for a technician to grab the end of the oblique tab for removal of the tab from the cannula 10. In yet another embodiment there can be no tab 14.

Further, the nasal sensor portion 308 of the thermal sensor assembly 300 has defined therein holes 32 and 34, which can receive the nasal prongs 307 of the cannula 10. The holes 32 and 34 define an alignment feature of the substrate 310 and of the thermal sensor assembly 300. The nasal prongs 307 define an alignment feature of the cannula 10. The alignment feature of the cannula (the prongs 307) cooperate with the holes 32 and 34 to align the thermal sensor assembly 300 to the cannula. As such, the thermal sensor assembly 300 is self aligning with respect to the cannula 10. That is, a technician placing the thermal sensor assembly 300 onto the cannula 10 only needs to place the nasal prongs 307 into the holes 32 and 34 and to join the thermal sensor assembly 300 to the cannula 10. By doing so, the nasal thermal sensors 16 and 18 are aligned to receive air from the nasal openings 306.

The substrate 310 also defines a substrate oral portion 314 which has the oral thermal sensor 22 formed thereon. The substrate oral portion 314 can have tabs 24 which can be used to secure the substrate oral section 314 to the cannula oral section 110.

The nasal thermal sensors 16 and 18, and the oral thermal sensor 22 can be thermocouple sensors, thermistor sensors, bead sensors, or any other suitable type of sensor that allows for the measurement of temperature. Additionally, the nasal thermal sensors 16 and 18, and the oral thermal sensor 22 can be made of thin deposits of electrically conductive ink. An electrically insulating, thermally conductive protective layer (e.g., a bio-compatible electrically insulating epoxy) can be formed over the nasal thermal sensors 16 and 18, the oral thermal sensor 22, and the electrically conductive traces 312 to allow proper temperature measurement of the air coming out of the patient and to avoid any extraneous electrical signal being picked up by the sensors and the conductive traces. An bio-compatible electrically insulating epoxy such as Loctite HYSOL M-31CL could be used.

The side of the substrate shown in the thermal sensor assembly 300 of FIG. 2 is the sensor side of the substrate. That is, the side of the substrate 310 that has the thermal sensors formed thereon.

The side of the substrate opposite to the sensor side (the backside) can have an adhesive layer portion secured thereto. The adhesive layer portion allows the thermal sensor assembly 300 to be secured to the cannula 10. The side of the substrate opposite to the sensor side can also have a stiffener secured thereto, to facilitate the electrical connection of the electrodes 28 to a measurement apparatus through an electrical connector and to protect the electrodes against excessive bending. FIG. 3 shows such a stiffener 38 formed at the tail portion 8 and an adhesive layer portion 36 formed at the sensor portion 308. As will be understood by the skilled worker, the adhesive layer portion 36 need not be applied over the entire backside of the sensor portion of the thermal sensor assembly 300. The stiffener 38 can be made of any suitable rigid or semi-rigid material such as, for example plastic. As another example, a double layer of substrate material, or a thicker layer of substrate material could be used as a stiffener. The stiffener 38 can be secured to the substrate 310 with any suitable adhesive or through any other suitable means.

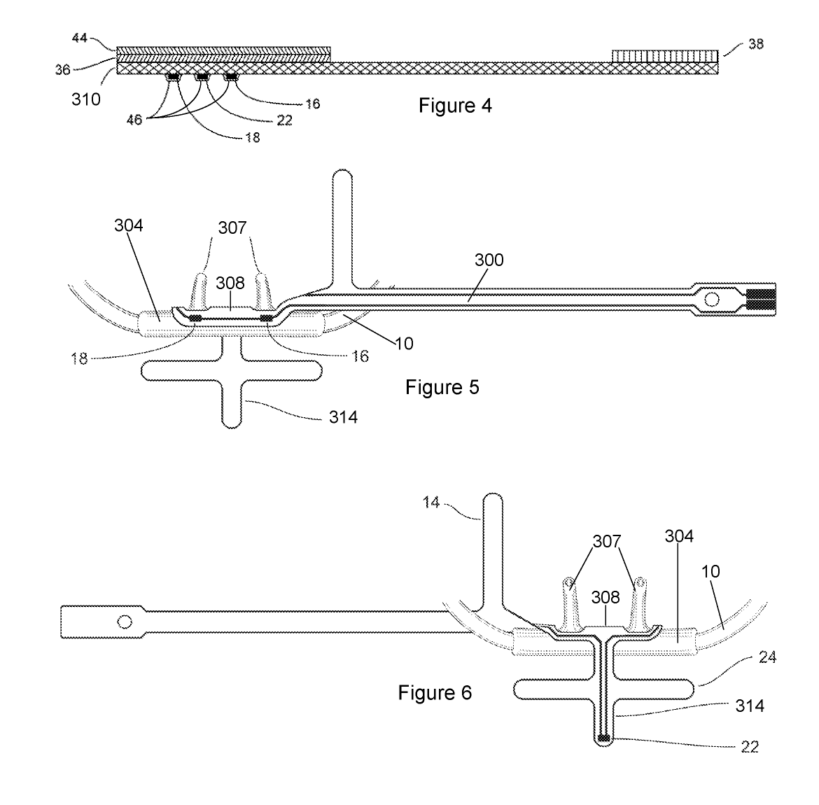

FIG. 4 shows a side view thermal sensor assembly 300, which is shown with the stiffener 38 secured to the substrate 310, the adhesive layer portion 36 formed on the substrate 310, and a peel-way backing 44 that protects the adhesive layer 36 until the thermal sensor assembly 300 is ready to be secured to the cannula 10. Also shown in FIG. 4 are the nasal thermal sensors 16 and 18, and the oral thermal sensor 22. Additionally, a layer of electrically insulating, thermally conductive material is shown, at reference numeral 46, formed over the nasal thermal sensors 16 and 18, and the oral thermal sensor 22. An example of material that can be used at 46 is Loctite® HYSOL™ M-31CL. Any other suitable material can be used.

The thermal sensor assembly 300 is such that, when secured to the cannula 10 and with the cannula being secured to a patient (individual), the nasal thermal sensors 16 and 18, and the oral thermal sensor 22 line-up with the nasal openings 306 and with the mouth of the patient 302. Further, the tab 14, which has the adhesive portion 36 formed thereon, facilitates the connection of the thermal sensor assembly 300 to the cannula 10 and can provide relief of strain applied at the tail portion 8. The thermal sensor assembly being secured to the cannula and the cannula being secured to the individual can be referred to as the thermal sensor assembly installed position or simply as the installed position.

To secure the thermal sensor assembly 300 to the cannula 10, the user (technician, clinician, etc.) first removes the peal-away backing 44 to expose the adhesive layer portion 36. The user then slides the nasal prongs 307 of the cannula into the holes 32 and 34 to begin securing the sensor portion 308 to the nasal portion 304 of the cannula 10 by adhering the sensor portion 308 to the cannula 10. This is shown in a front view at FIG. 5, and in a rear view at FIG. 6.

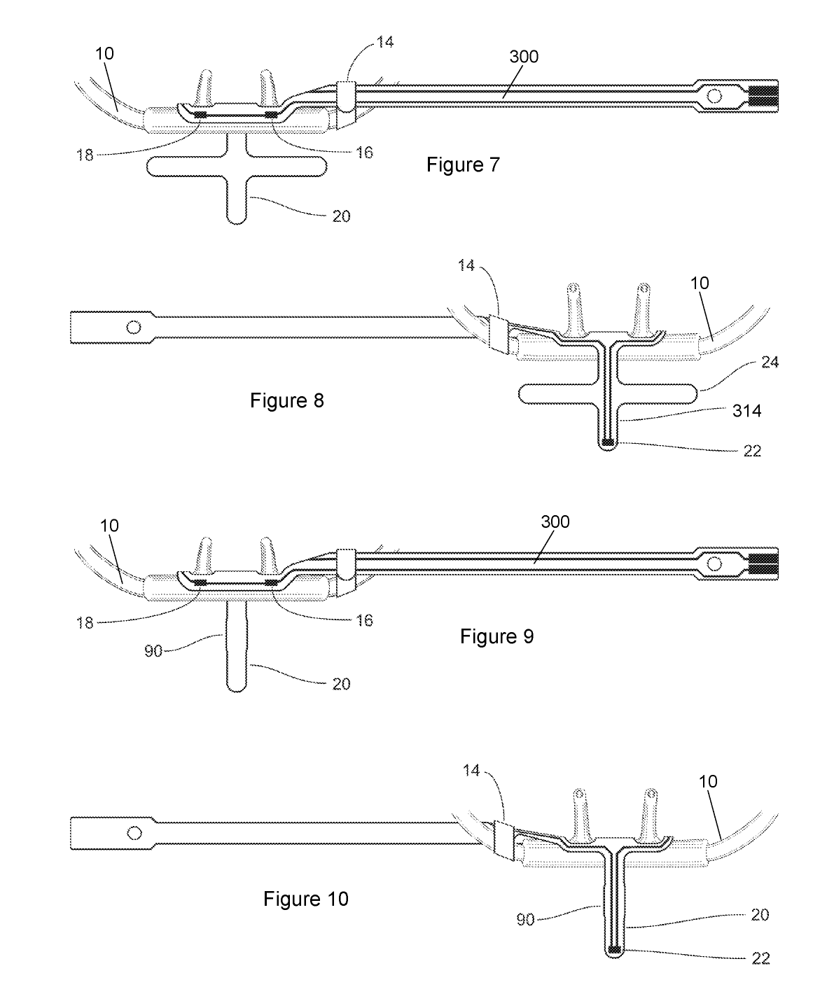

Subsequently, the user can wrap the tab 14 to the cannula 10 and back onto itself, as show in front and rear views at FIGS. 7 and 8 respectively.

If the thermal sensor assembly 300 is used with a nasal-only cannula, the tabs 24 may be cut off as shown in front and rear views at FIGS. 9 and 10 respectively. As such, the oral thermal sensor 22 dangles from the cannula 10 and faces the mouth of the patient 302. The electrically insulating, thermally conductive material 46 mitigates interference from any contact of the oral thermal sensor 22 with the patient.

FIGS. 11 and 12 show respectively a front view and a rear view of the thermal sensor assembly 300 being secured to an oronasal cannula 10 prior to the tab 14 being secured to the cannula 10. FIG. 11 also shows an oral section 110 of the cannula 10 and FIG. 12 an opening 120 for the oral pressure wave. The thermal sensor assembly 300 can be dimensioned such that, when the thermal sensor assembly is secured to the oronasal cannula 10, the oral thermal sensor 22 does not occlude the opening 120. For example, in the view shown at FIG. 12, the oral thermal sensor 22 lies above the opening 120 and does not occlude the opening 120.

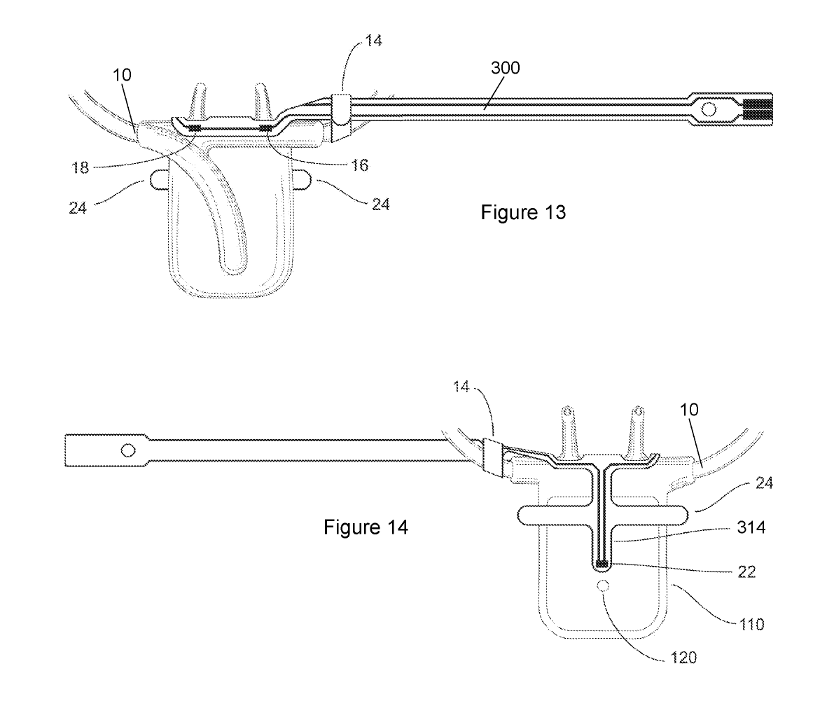

FIGS. 13 and 14 show respectively a front view and a rear view of the thermal sensor assembly 300 secured to an oronasal cannula 10 with the tab 14 wrapped around the cannula 10.

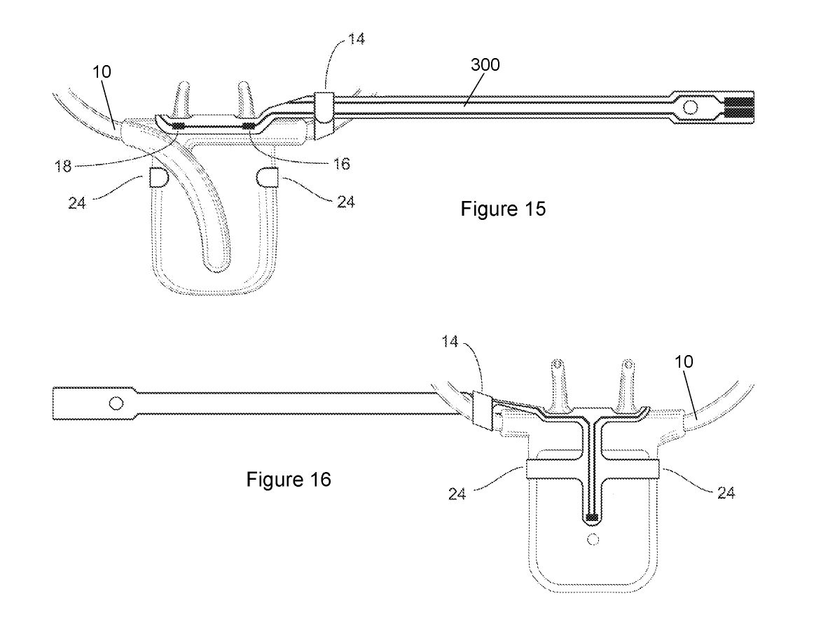

FIGS. 15 and 16 show respectively a front view and a rear view of the thermal sensor assembly 300 secured to an oronasal cannula 10 with the tabs 24 wrapped adhered to the cannula 10. Although the tabs 24 are shown extending parallel to the intermediate portion 6 shown at FIG. 2, this need not be the case. The tabs 24 can be at any suitable angle to the intermediate portion 6 without departing from the scope of the present disclosure.

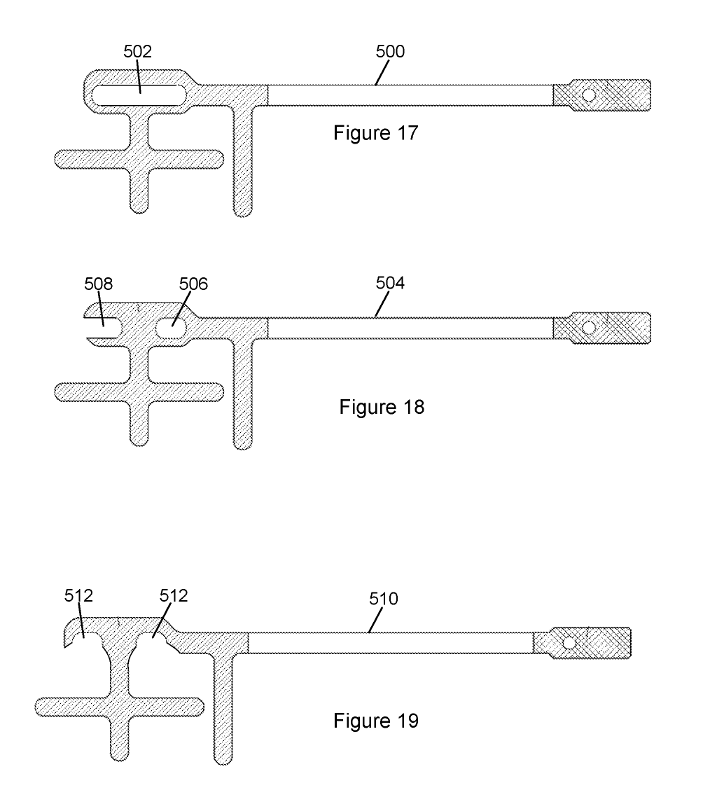

FIG. 17 shows another embodiment of a thermal sensor assembly of the present disclosure. The thermal sensor assembly 500 of FIG. 17 has one elongated opening 502, which can also be referred to as an alignment aperture or feature, that can fit over nasal prongs of a cannula to align and secure the thermal sensor assembly 500 the cannula in question.

FIG. 18 shows another embodiment of a thermal sensor assembly of the present disclosure. The thermal sensor assembly 504 of FIG. 18 has one opening 506, and a slot 508, both of which can also be referred to as alignment apertures or alignment features, that can fit over nasal prongs of a cannula to align and secure the thermal sensor assembly 504 the cannula in question.

FIG. 19 shows another embodiment of a thermal sensor assembly of the present disclosure. The thermal sensor assembly 510 of FIG. 19 has recesses 512, both of which can also be referred to as alignment apertures or alignment features, that can fit over nasal prongs of a cannula to align and secure the thermal sensor assembly 510 the cannula in question.

The views of the thermal sensor assemblies of FIGS. 17 to 19 are bottom views. That is, the thermal sensors of the thermal sensor assemblies are on the opposite side of the side shown in FIGS. 17 to 19.

In the preceding description, for purposes of explanation, numerous details are set forth in order to provide a thorough understanding of the embodiments. However, it will be apparent to one skilled in the art that these specific details are not required.

The above-described embodiments are intended to be examples only. Alterations, modifications and variations can be effected to the particular embodiments by those of skill in the art without departing from the scope, which is defined solely by the claims appended hereto.

What is claimed is:

1. A thermal sensor assembly to measure a temperature of air expelled by an individual, the thermal sensor assembly to be secured to a cannula having nasal prongs, the thermal sensor assembly secured to the cannula and the cannula secured to the individual defining an installed position, the assembly comprising:
a substrate having a sensor portion, the substrate further having a sensor side and a backside, the backside being opposite the sensor side, the sensor portion defining an alignment aperture, the alignment aperture to receive at least one of the nasal prongs to align the thermal sensor assembly to the cannula;
at least one nasal thermal sensor formed on the sensor side of the substrate and at the sensor portion of the substrate, the at least one nasal thermal sensor being adjacent to the alignment aperture, the at least one thermal sensor to sense, in the installed position, a temperature of air expelled through a nasal opening of the individual; and
an adhesive layer formed on the backside of the substrate and at the sensor portion of the substrate, the adhesive layer to adhere the sensor portion of the substrate to the cannula.

2. The thermal sensor assembly of claim 1 wherein the substrate further has a tab to secure, in the installed position, the thermal sensor assembly to the cannula.

3. The thermal sensor assembly of claim 2 wherein the backside of the tab has an adhesive formed thereon.

4. The thermal sensor assembly of claim 1 wherein the sensor portion defines two alignment apertures, each alignment aperture to receive a single nasal prong of the cannula.

5. The thermal sensor assembly of claim 2 wherein the substrate further has:
a tail portion; and
an intermediate portion bridging the sensor portion and the tail portion.

6. The thermal sensor assembly of claim 6 wherein the tab extends from the intermediate portion and away from the intermediate portion.

7. The thermal sensor assembly of claim 1 wherein the alignment aperture is to receive both nasal prongs.

8. The thermal sensor assembly of claim 1 wherein the sensor portion has an oral sensor portion, the oral sensor portion having formed thereon an oral thermal sensor to sense, in the installed position, a temperature of air expelled from the mouth of the individual.

9. The thermal sensor assembly of claim 8 wherein the oral sensor portion has at least one oral sensor portion tab to secure the oral sensor portion to the cannula.

10. The thermal sensor assembly of claim 8 further comprising an electrically insulating, thermally conductive material formed over at least one of the at least one nasal thermal sensor and the oral thermal sensor.

11. The thermal sensor assembly of claim 8 wherein the sensor side of the substrate has formed thereon at least on electrical conductor to electrically connect the at least one nasal thermal sensor and the oral thermal sensor.

12. The thermal sensor assembly of claim 1 wherein the substrate is made of an electrically insulating material.

13. The thermal sensor assembly of claim 12 wherein the electrically insulating material is material or polyester.

14. The thermal sensor assembly of claim 12 wherein the electrically insulating material is flexible.

15. The thermal sensor assembly of claim 1 wherein, in the installed position, a section of the substrate on which the at least one nasal thermal sensors are formed lies between the cannula and the at least one nasal thermal sensor.

16. The thermal sensor assembly of claim 5 wherein the tail portion has electrodes formed thereon, the electrodes to electrically connect the thermal sensor assembly to a measurement apparatus.

17. The thermal sensor assembly of claim 16 wherein the tail portion defines a hole, the hole to receive a cooperating element of an electrical connector to secure the tail portion to the electrical connector.

18. The thermal sensor assembly of claim 5 further comprising a stiffener secured the tail portion.

19. A thermal sensor assembly to measure a temperature of air expelled by an individual, the thermal sensor assembly to be secured to a cannula having nasal prongs, the thermal sensor assembly secured to the cannula and the cannula secured to the individual defining an installed position, the assembly comprising:
a substrate having a sensor portion, the substrate further having a sensor side and a backside, the backside being opposite the sensor side, the sensor portion defining an alignment feature, the alignment feature to receive at least one of the nasal prongs to align the thermal sensor assembly to the cannula;
at least one nasal thermal sensor formed on the sensor side of the substrate and at the sensor portion of the substrate, the at least one nasal thermal sensor being adjacent to the alignment feature, the at least one thermal sensor to sense, in the installed position, a temperature of air expelled through a nasal opening of the individual; and
an adhesive layer formed on the backside of the substrate and at the sensor portion of the substrate, the adhesive layer to adhere the sensor portion of the substrate to the cannula.
